SEQUENCE LISTING (8 sequences)

SEQ ID NO: 1 - DNA, 24 bp, Artificial Sequence
  Synthetic Construct
aaacagagtagcagcgcagactgc

SEQ ID NO: 2 - DNA, 26 bp, Artificial Sequence
  Synthetic Construct
ggatctctaaaactagaggcttggtg

SEQ ID NO: 3 - DNA, 21 bp, Artificial Sequence
  Synthetic Construct
ctgagtccgaatcaggtgcag

SEQ ID NO: 4 - DNA, 21 bp, Artificial Sequence
  Synthetic Construct
atccatgggaagatgttctgg

SEQ ID NO: 5 - DNA, 23 bp, Artificial Sequence
  Synthetic Construct
tgggtacatttgatctgactgga

SEQ ID NO: 6 - DNA, 23 bp, Artificial Sequence
  Synthetic Construct
ctcaaaggtgacttcaatctggg

SEQ ID NO: 7 - DNA, 21 bp, Artificial Sequence
  Synthetic Construct
gttggttttcggaactgaggc

SEQ ID NO: 8 - DNA, 20 bp, Artificial Sequence
  Synthetic Construct
gtcggcatcgtttatggtcg

(19) United States
(12) Patent Application Publication
(10) Pub. No.: US 2019/0038597 A1
(43) Pub. Date: Feb. 7, 2019

(54) PREVENTION OF VASCULAR AND CARDIAC FIBROSIS VIA IRE1ALPHA INHIBITION

(71) Applicant: McMaster University, Hamilton, CA (US)

(72) Inventors: Jeffrey Dickhout, Dundas (CA); Kjetil Ask, Ancaster (CA)

(21) Appl. No.: 16/052,948

(22) Filed: Aug. 2, 2018

Related U.S. Application Data
(60) Provisional application No. 62/540,628, filed on Aug. 3, 2017.

(51) Int. Cl. A61K 31/381; A61K 31/167; A61K 31/4433; A61K 31/37; A61K 31/17; A61K 31/12; A61K 31/496; A61K 31/436; A61K 31/4985; A61K 31/7064; A61K 31/404; A61K 31/506; A61P 9/00

(52) U.S. Cl. CPC A61K 31/381; A61K 31/167; A61K 31/4433; A61K 31/37; A61K 31/17; A61K 31/12; A61P 9/00; A61K 31/436; A61K 31/4985; A61K 31/7064; A61K 31/404; A61K 31/506; A61K 31/496

(57) ABSTRACT

Provided herein are methods and compositions for treating and/or reducing the risk of developing a condition associated with fibrosis and/or collagen deposition in a subject. In one embodiment, the method comprises administering an effective amount of an IRE1α inhibitor to the subject; wherein the subject is identified as having a risk of developing and/or a need for treatment of the condition associated with fibrosis and/or collagen deposition.

### CROSS REFERENCE TO RELATED APPLICATIONS

The present application claims priority from U.S. provisional application No. 62/540,628 filed on Aug. 3, 2017, which is hereby incorporated by reference in its entirety.

### INCORPORATION OF SEQUENCE LISTING

A computer readable form of the Sequence Listing “3244-P53770US01_SequenceListing.txt” (4,096 bytes), submitted via EFS-WEB and created on Jul. 31, 2018, is herein incorporated by reference.

### FIELD

The present disclosure relates to treating and/or reducing risk of conditions associated with fibrosis and collagen deposition. In particular, the presently-disclosed subject matter relates to inhibiting IRE1α to treat and/or reduce risk of such conditions.

### BACKGROUND

Arterial stiffening contributes to the development of systolic hypertension through alterations in central hemodynamics¹. Increased arterial stiffness is associated with end-organ damage in the heart, kidneys and brain²⁻⁸. It is also associated with an increased risk of cardiovascular events and mortality⁹. Arterial stiffening can be caused by the fibrotic remodelling of the vessel wall, in which collagen content increases while elastin fibers become fragmented and degraded¹⁰.

Elevated systolic blood pressure (SBP) increases the left ventricular load, eventually leading to left ventricular hypertrophy¹¹. The increased oxygen demand of the hypertrophied tissue, coupled with the decreased coronary perfusion during diastole, results in ischemia and an increased risk of heart failure²⁻⁴. Cardiomyocyte hypoxia leads to scar tissue deposition.

Vascular and cardiac fibrosis involve cells that generate excessive amounts of extracellular matrix (ECM) proteins, primarily Type I collagen. Transforming growth factor-β(TGF-β) is a major profibrotic cytokine that stimulates this process and so is Angiotensin II. These molecules stimulate collagen synthesis in vascular smooth muscle cells (VSMCs) and fibroblasts.

Fibrotic VSMCs and fibroblasts experience an increase in protein translation and secretion. To augment protein-folding capacity, the unfolded protein response (UPR) is activated in these cells. IRE1α in particular has been shown to contribute to this adaptive response by increasing the expression of protein folding chaperones. IRE1α contains an endonuclease domain that mediates the splicing of a 26 nucleotide intron from the mRNA of the transcription factor X-box binding protein 1 (XBP1)²⁴. The spliced form of XBP1 (sXBP1) binds to UPR elements to upregulate the transcription of components involved in the UPR²⁴. These transcriptional targets include chaperones such as GRP78, GRP94, and protein disulfide isomerase (PDI), all of which have been shown to participate in the folding and assembly of procollagen molecules into a triple helix^{23,24-29}.

### SUMMARY

The present inventors have shown that the IRE1α pathway is required for collagen synthesis from vascular smooth muscle cells and fibroblasts and that it contributes to vascular stiffening and end-organ damage in hypertension. In particular, the present inventors have shown that inhibition of IRE1α endonuclease activity reduces the expression of chaperones involved in the biosynthesis of collagen, which hinders the proper folding and assembly of the collagen triple helix preventing vascular stiffening and end organ damage, for example, of the heart.

Accordingly, in one aspect, provided herein is a method of reducing fibrosis and/or collagen deposition in a subject, comprising administering an effective amount of an IRE1α inhibitor to the subject in need thereof.

In another aspect, provided herein are methods of treating and/or reducing the risk of developing a condition associated with fibrosis and/or collagen deposition in a subject, comprising: administering an effective amount of an IRE1α inhibitor to the subject in need thereof.

In an embodiment, the condition associated with fibrosis and/or collagen deposition is aortic stiffening/hypertension, vascular fibrosis, vascular disease, cardiovascular disease, diastolic dysfunction, heart failure with preserved ejection fraction, arteriosclerosis, restenosis associated with vascular surgery (including but not limited to stenting or angioplasty), and/or myocardial fibrosis. In a particular embodiment, the condition associated with fibrosis and/or collagen deposition is aortic stiffening/hypertension. In another embodiment, the condition associated with fibrosis and/or collagen deposition is interstitial renal fibrosis.

In an embodiment, the inhibitor is administered orally, topically or by local injection.

In an embodiment, the subject is human.

In an embodiment, the IRE1α inhibitor targets IRE1α expression. In another embodiment, the IRE1α inhibitor targets IRE1α endonuclease activity.

In an embodiment, the IRE1α inhibitor is a compound listed in Table 1 or an analog thereof. In one embodiment, the IRE1α inhibitor is STF-083010, MKC-3946, 4μ8C, 3-methoxy-6-bromosalicylaldehyde, toyocamycin or analogs thereof. In a particular embodiment, the IRE1α inhibitor is 4μ8C or an analog thereof. In another particular embodiment, the IRE1α inhibitor is S′TF-083010 or an analog thereof.

In another embodiment, the IRE1α inhibitor is an antisense oligonucleotide molecule directed to IRE1a. In another embodiment, the IRE1α inhibitor is an shRNA or siRNA molecule that inhibits expression of IRE1a. In yet another embodiment, the IRE1α inhibitor is an antibody specific to IRE1a.

In yet another embodiment, the IRE1α inhibitor comprises a CRISPR-Cas9 complex to modulate the transcriptional activity at the endogenous IRE1α locus.

Other features and advantages of the present disclosure will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Embodiments are described below in relation to the drawings in which:

FIG. 1 demonstrates that 4μ8c and STF-083010 block IRE1α mediated XBP1 splicing. VSMCs were treated with tunicamycin (1 μg/ml) and either 4μ8c (30 μM) or STF-083010 (60 μM) for 6 h or 24 h. (A) Agarose gel for assessment of XBP1 splicing after 6 h of treatment with tunicamycin and densitometric quantification. (B) Western blot for assessment of ER chaperone expression after 24 h tunicamycin treatment with or without inhibition of IRE1α by 4μ8c. Data is presented as mean±SEM. *, p<0.05 vs. Vehicle; #, p<0.05 vs. Tm.

FIG. 2 demonstrates that IRE1α inhibition prevents collagen deposition by VSMCs. VSMCs were cultured in DMEM/F12 containing 1% FBS. Confluent cells were treated with L-ascorbic acid-2-phosphate (100 μg/ml) and either TGF-β1 (5 ng/ml) or Ang II (1 μM). (A) Agarose gel for assessment of TGF-β1-induced XBP1 splicing in VSMCs after 24 h. (B) Picro-Sirius Red assay for measurement of collagen deposition induced by combined TGF-β1 and AA2P treatment after 72 h, and inhibition by 4μ8c and STF-083010. (C) Western blot for assessment of collagen chaperone expression after treatment of TGF-β1 and 4μ8c for 48 h. *, p<0.05. (D) Measurement of Ang II-induced collagen production and inhibition by 4μ8c and STF-083010 after 72 h.

FIG. 3 demonstrates that IRE1α inhibition prevents collagen deposition by renal fibroblasts. Rat renal fibroblasts were treated with L-ascorbic acid-2-phosphate, TGF-β1 and 4μ8c for 72 h. Collagen production was measured by a Picro-Sirius Red assay. **, p<0.01.

FIG. 4 demonstrates that 4μ8c does not affect blood pressure in L-NAME-treated SHRs. 12-14 week old male SHRs were implanted with radiotelemetry devices and randomized to receive vehicle, L-NAME (50 mg/L) or L-NAME and 4μ8c (2.5 mg/kg/day i.p.) for 18 days. Systolic (A), diastolic (B), and heart rate (C) were monitored. Data is presented as mean±SEM. *, p<0.05 vs. NT; #, p<0.05 vs. L-NAME. N≥4 per group.

FIG. 5 demonstrates that 4μ8c reduces vascular stiffening and fibrosis induced by L-NAME. 12-14 week old male SHRs were randomized to receive vehicle, L-NAME (50 mg/L) or L-NAME and 4μ8c (2.5 mg/kg/day i.p.) for 18 days. Aortas and carotid arteries were removed at sacrifice for mechanical and structural analysis. (A) Picro-Sirius Red staining of aorta and carotid artery sections (B) Determination of soluble collagen content in aorta and carotid arteries by Picro-Sirius Red-based assay. Stress-strain curves (C,E), elastic modulus-stress lines (D,F) and the slope of the elastic modulus-stress lines (G,H) were calculated. Data is presented as mean±SEM. *, p<0.05 vs. NT; #, p<0.05 vs. L-NAME. N≥4 per group; 2-5 segments of the thoracic aorta were analyzed per animal. In C to F, lines of best fit are bound by 95% confidence intervals (dotted lines). *, p<0.05.

FIG. 6 shows the effect of 4μ8c on aortic collagen chaperone expression in L-NAME-treated SHRs. 12-14 week old male SHRs were randomized to receive vehicle, L-NAME (50 mg/L) or L-NAME and 4μ8c (2.5 mg/kg/day i.p.) for 18 days. Total RNA was extracted from flash frozen aortic tissues. (A) qRT-PCR was used to assess relative mRNA expression of spliced XBP1 and GRP78. 18S was used as a housekeeping gene. (B) Protein levels of PDI and GRP78 were assessed by western blotting and normalized to β-actin. Data is presented as mean±SEM. *, p<0.05 vs. NT. #, p<0.05 vs L-NAME.

FIG. 7 demonstrates that 4μ8c reduces cardiac fibrosis in L-NAME-treated SHRs. 12-14 week old male SHRs were randomized to receive vehicle, L-NAME (50 mg/L) or L-NAME and 4μ8c (2.5 mg/kg/day i.p.) for 18 days. (A) Cardiac hypertrophy measured using heart weight to body ratio. (B) Quantification of Picro-Sirius Red-stained area in heart tissue. (C) Picro-Sirius Red staining of transverse heart sections. Bar: 4×, 500 μm; 20×, 200 μm. Data is presented as mean±SEM. *, p<0.05 vs. NT. #, p<0.05 vs L-NAME.

### DETAILED DESCRIPTION

### Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise. For example, an embodiment including “an inhibitor” should be understood to present certain aspects with one substance or two or more additional substances.

In embodiments comprising an “additional” or “second” component, such as an additional or second inhibitor, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “subject” as used herein includes all members of the animal kingdom including mammals such as a mouse, a rat, a dog and a human.

The expression a “therapeutically effective amount”, “effective amount” or a “sufficient amount” of a compound refers to a quantity sufficient to, when administered to the subject, including a mammal, for example a human, effect beneficial or desired results, including clinical results, and, as such, a “therapeutically effective amount” or an “effective amount” depends upon the context in which it is being applied. For example, in the context of treating fibrosis, it is an amount of the compound sufficient to achieve such treatment of the fibrosis as compared to the response obtained without administration of the compound. The amount of a given compound or composition that will correspond to an effective amount will vary depending upon various factors, such as the given drug or compound, the pharmaceutical formulation, the route of administration, the type of disease or disorder, the identity of the subject or host being treated, and the like, but can nevertheless be routinely determined by one skilled in the art. Also, as used herein, a “therapeutically effective amount” or “effective amount” of a compound is an amount which inhibits, or reduces the fibrosis (e.g., reducing the amount of collagen deposition or inhibiting vascular stiffness) in a subject as compared to a control.

As used herein, and as well understood in the art, “treatment” or “treating” is an approach for obtaining beneficial or desired results, including clinical results. Beneficial or desired clinical results can include, but are not limited to, inhibition of fibrosis or decrease of fibrosis by at least about 10%, at least about 20%, at least about 30%, at least about 40%, at least about 50%, at least about 60%, at least about 70%, at least about 80% or at least about 90% greater than an untreated control cell. “Treatment” also means alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term “administered” or “administering” as used herein means administration of a therapeutically effective amount of a compound to a cell either in vitro (e.g. a cell culture) or in vivo (e.g. in a subject).

### Methods of the Disclosure

In one aspect, provided herein is a method of reducing fibrosis and/or collagen deposition in a subject, comprising administering an effective amount of an IRE1α inhibitor to the subject in need thereof.

The phrase “reducing fibrosis” as used herein refers to preventing or reducing extracellular matrix fibers from accumulating in tissue. The term “extracellular matrix” as used herein refers to fibers that are typically secreted from a cell that reside in the extracellular space and include without limitation, collagen and fibronectin.

Inhibiting fibrosis as used herein refers to a decrease of at least 5%, 10%, 20%, 30%, 40%, 50%, 60%, 70%, 80% or more of extracellular matrix accumulation compared to an untreated control or reference value.

In another aspect, provided herein are methods of treating and/or reducing the risk of developing a condition associated with fibrosis and/or collagen deposition in a subject, comprising: administering an effective amount of an IRE1α inhibitor to the subject in need thereof.

In an embodiment, the condition associated with fibrosis and/or collagen deposition is aortic stiffening/hypertension, vascular fibrosis, vascular disease, cardiovascular disease, diastolic dysfunction, heart failure with preserved ejection fraction, arteriosclerosis, restenosis associated with vascular surgery (including but not limited to stenting or angioplasty), and/or myocardial fibrosis. In a particular embodiment, the condition associated with fibrosis and/or collagen deposition is aortic stiffening/hypertension. In another embodiment, the condition associated with fibrosis and/or collagen deposition is renal interstitial fibrosis. In another embodiment, the condition associated with fibrosis and/or collagen deposition is dilated cardiomyopathy.

In an embodiment, the subject is human.

The term “inhibitor” and its derivatives, as used herein, refers to an agent that reduces, decreases, or otherwise blocks expression or activity of its target, and includes any substance that is capable of inhibiting the expression or activity of the target and includes, without limitation, small molecules, antisense oligonucleotide molecules (antisense nucleic acid molecules), siRNAs or shRNAs, aptamers, proteins, antibodies (and fragments thereof), gene editing agents and other substances directed at the target expression or activity.

The term “IRE1a” as used herein refers to inositol-requiring enzyme-1-alpha and can be from any source, including human (Genbank Accession Nos: AAI30408.1 and NM_001433.4).

The term “IRE1α activity and/or expression” and its derivatives, as used herein, refers to transcriptional, translational, functional and/or enzymatic activity, and/or protein and/or mRNA expression, involving IRE1a. IRE1α activity includes endonuclease activity and the consequent expression of chaperones involved in the biosynthesis of collagen.

The term “IRE1α inhibitor” and its derivatives, as used herein, refers to an agent whose presence, level, state and/or form correlates with a reduction in IRE1α level and/or activity. That is, observed IRE1α level and/or activity is detectably lower in the presence of the agent (or when the agent is at a particular level, or in a particular state or form) as compared to its absence and/or as compared to a comparable reference.

Accordingly, in an embodiment, the IRE1α inhibitor is an inhibitor of IRE1α expression and/or activity.

IRE1α inhibitors are known in the art and Table 1 lists some of the known inhibitors as well as the structures, CAS numbers and formulae. In an embodiment, the IRE1α inhibitor is an inhibitor listed in Table 1. In one embodiment, the IRE1α inhibitor is a STF-083010, MKC-3946, 4μ8C, 3-methoxy-6-bromosalicylaldehyde, toyocamycin or analogs thereof. In a particular embodiment, the IRE1α inhibitor is 4μ8C or an analog thereof. In another particular embodiment, the IRE1α inhibitor is STF-083010 or an analog thereof.

Aptamers are short strands of nucleic acids that can adopt highly specific 3-dimensional conformations. Aptamers can exhibit high binding affinity and specificity to a target molecule. These properties allow such molecules to specifically inhibit the functional activity of proteins and are included as agents that inhibit IRE1α activity. Accordingly, in another embodiment, the inhibitor is an aptamer that inhibits IRE1α activity.

In another embodiment, the IRE1α inhibitor is an antibody specific to IRE1a, wherein the antibody interferes with the RNA splicing domain of the IRE1α protein.

The term “antibody” as used herein is intended to include monoclonal antibodies, polyclonal antibodies, chimeric and humanized antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The inhibitors described herein may also contain or be used to obtain or design “peptide mimetics”. For example, a peptide mimetic may be made to mimic the function of an inhibitor. “Peptide mimetics” are structures which serve as substitutes for peptides in interactions between molecules (Morgan and Gainor, 1989). Peptide mimetics include synthetic structures, which may or may not contain amino acids and/or peptide bonds but retain the structural and functional features. Peptide mimetics also include molecules incorporating peptides into larger molecules with other functional elements. Peptide mimetics also include peptoids, oligopeptoids (Simon et al., 1972) and peptide libraries containing peptides of a designed length representing all possible sequences of amino acids corresponding to an inhibitor peptide disclosed herein.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of the secondary structures of the proteins described herein. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

The term “nucleic acid molecule” and its derivatives, as used herein, are intended to include unmodified DNA or RNA or modified DNA or RNA. For example, the nucleic acid molecules or polynucleotides of the disclosure can be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, the nucleic acid molecules can be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning.

The term “siRNA” refers to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

Accordingly, in another embodiment, the inhibitor is at least one siRNA or shRNA molecule that inhibits expression of IRE1a.

The term “antisense oligonucleotide” or “antisense nucleic acid” and its derivatives, as used herein, mean a nucleotide sequence that is complementary to its target transcription product. The nucleic acid can comprise DNA, RNA or a chemical analog that binds to the messenger RNA produced by the target gene. Binding of the antisense oligonucleotide prevents translation and thereby inhibits or reduces target protein expression. Antisense oligonucleotide molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced. Accordingly, in one embodiment, the inhibitor is at least one antisense oligonucleotide molecule that is complementary to IRE1a, for example human IRE1a.

The term “gene” and its derivatives, as used herein, refer to a genomic DNA sequence that comprises a coding sequence associated with the production of a polypeptide or polynucleotide product (e.g., rRNA, tRNA).

The term “gene editing agents” and its derivatives, as used herein, refer to Cre recombinases, CRISPR-Cas9 complex, TALE transcriptional activators, Cas9 nucleases, nickases, transcriptional regulators or combinations thereof. In an embodiment, the IRE1α inhibitor comprises a CRISPR-Cas9 complex to modulate the transcriptional activity at the endogenous IRE1α locus. In a specific embodiment, the CRISPR-Cas9 complex comprises Cas9 nuclease complexed with a synthetic single guide RNA (sgRNA) that targets IRE1α.

The inhibitor for use in the methods of the present disclosure may be formulated into a pharmaceutical composition, such as by mixing with a suitable excipient, carrier, and/or diluent, by using techniques that are known in the art. For example, the inhibitor can be used or administered in the form of tablets, capsules, ovules, elixirs, solutions or suspensions, which may contain flavouring or colouring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the inhibitor may be combined with various sweetening or flavouring agents, colouring matter or dyes, with emulsifying and/or suspending agents and with diluents such as water, ethanol, propylene glycol and glycerin, and combinations thereof.

As used herein, the term “pharmaceutically acceptable carrier” is intended to include any and all solvents, dispersion media, coatings, isotonic and absorption delaying agents, and the like, compatible with pharmaceutical use or administration. Suitable carriers are described in the most recent edition of Remington's Pharmaceutical Sciences, a standard reference text in the field, which is incorporated herein by reference. Optional examples of such carriers or diluents include, but are not limited to, water, saline, ringer's solutions, dextrose solution, and 5% human serum albumin.

In one embodiment, the active ingredient is prepared with a carrier that will protect it against rapid elimination from the body, such as a sustained/controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

A pharmaceutical composition is formulated to be compatible with its intended route of use or administration. The use or administration of inhibitor to a subject comprises ingestion, inhalation, or injection. The route of injection includes but not limited to intradermal, subcutaneous, intramuscular, intravenous, intraosseous, intraperitoneal, intrathecal, epidural, intracardiac, intraarticular, intracavernous, intravitreal, intracerebral, intracerebroventricular, or intraportal. In an embodiment, the inhibitor is administered orally, topically or by local injection.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure.

### EXAMPLES

### Experimental Methods and Procedures

### Cell Culture

VSMCs were isolated from the aortas of 5-week old WKY rats using the explant method²⁹. The thoracic aorta was removed from the animal and placed in ice-cold 5× Antibiotics-Antimycotics solution (ThermoFisher). The adventitia was gently removed and the aorta was cut longitudinally. The endothelial layer was removed by gentle scraping with forceps. The aorta was cut into 3 mm² squares and placed on a 6 well plate in DMEM/F12 medium containing 10% fetal bovine serum (FBS). Explants were incubated undisturbed in a 5% CO₂ incubator at 37° C. for 1 week and then passaged. Sprague-Dawley renal fibroblasts were purchased (Cell Biologics) and cultured in low glucose DMEM/F12 medium containing 10% FBS, streptomycin (100 μgimp, and penicillin (100 U/ml, Invitrogen, Burlington, ON). Cells between passages 5 and 10 were used for experiments. Cells were cultured in medium containing 1% FBS overnight before treatment.

### Animals

12-14 week old male SHRs were used to determine the effect of pharmacological IRE1α inhibition on L-NAME-induced vascular stiffening. They were maintained on a 12-hour light-dark cycle with rat chow and water ad libitium. Animals were implanted with radio-telemetry devices (Data Science International, St. Paul, Minn., USA) one week before the start of the study. Animals were randomized into one of three groups (n=6 per group): 1) No treatment, 2) L-NAME (50 mg/L in drinking water, Sigma-Aldrich), or 3) L-NAME+4μ8c (2.5 mg/kg/day i.p., Millipore). 4μ8c was administered in a vehicle of 10% DMSO, 10% Tween-80 and 80% normal saline. Animals in Group 2 received daily injections of the vehicle. All animals were fed a normal chow diet. At 18 days, animals were sacrificed, organs were harvested and the aorta was collected for mechanical and structural analysis. All animal work was performed according to the guidelines approved by the McMaster University Animal Research Ethics Board.

### XBP1 Splicing Assay

Total RNA was extracted from cultured cells and rat tissues using the TRIzol reagent (ThermoFisher) according to manufacturer's instructions. RNA concentration and purity were measured using a Nanodrop ND-1000 spectrophotometer (NanoDrop Technologies, Inc.). RNA was reverse transcribed using the High-Capacity cDNA Reverse Transcription Kit (Applied Biosystems) according to manufacturer's instructions. For qualitative assessment of XBP1 splicing, cDNA was amplified using recombinant Taq polymerase (Invitrogen) and primers for total)(BPI: Forward 5′-AAACAGAGTAGCAGCGCAGACTGC-3 ‘ (SEQ ID NO:1), Reverse 5’-GGATCTCTAAAACTAGAGGCTTGGTG-3′ (SEQ ID NO:2). PCR products were digested with PstI for 1 h at 37° C., separated on a 2% agarose gel, and visualized by Safe-Red (ABM, Inc.) and the ChemiDoc XRS+ system (BioRad).

qRT-PCR Analysis

For qRT-PCR, cDNA was amplified using the Fast SYBR Green Master Mix (ThermoFisher) and analyzed with 7500 Software. The primers used were as follows: spliced XBP1 (Forward 5′-CTGAGTCCGAATCAGGTGCAG-3′ (SEQ ID NO:3), Reverse 5′-ATCCATGGGAAGATGTTCTGG-3′ (SEQ ID NO:4)), GRP78/BiP (Forward 5′-TGGGTACATTTGATCTGACTGGA-3′ (SEQ ID NO:5), Reverse 5′-CTCAAAGGTGACTTCAATCTGGG-3′ (SEQ ID NO:6)), and 18S (Forward 5′-GTTGGTTTTCGGAACTGAGGC-3′ (SEQ ID NO:7), Reverse 5′-GTCGGCATCGTTTATGGTCG-3′ (SEQ ID NO:8)). 18S was used as an internal control for normalization.

### Immunoblotting

Cells were lysed in 1× Laemmli buffer and protein content was quantified using the BioRad DC Protein Assay (BioRad, Mississauga, Canada). Proteins were separated by SDS-PAGE under reducing conditions on a 7.5% gel and transferred to a PVDF membrane using the Trans-Blot Turbo Semi-Dry Transfer system (Bio-Rad, Richmond, Calif., USA). Membranes were blocked with 5% milk and incubated with primary antibodies: β actin (A-2228, 1:5000, Sigma-Aldrich, St. Louis, Mo., USA), KDEL (SPA-827, 1:1000, Stressgen, Victoria, Canada) and PDI (SPA-891, 1:1000, Enzo, Farmingdale, N.Y., USA). Membranes were then washed in TBST and incubated with a horseradish peroxidase-conjugated secondary antibody (Bio-Rad), followed by development with ECL Western Blotting Detection Reagents (GE Healthcare, Mississauga, Canada). Densitometric analysis was performed with Image-Lab Software (Bio-Rad) and band intensities were normalized against β-actin.

### Sirius Red Spectrophotometric Assay

Collagen synthesis was measured using a Picro-Sirius Red-based assay as previously described³⁰. Briefly, cells cultured on a 96 well plate were fixed in methanol for 10 minutes at −20° C. Cells were washed once with PBS and incubated with Picrosirius Red stain (0.1% Direct Red 80 in saturated picric acid) for 1 h at room temperature. After the staining solution was removed, the cells were washed three times with 0.1% acetic acid and then imaged. The dye was then eluted with 0.1 N NaOH for 10 minutes on a rocking platform. Absorbance at 540 nm was measured using a Spectramax Plus Microplate Reader (Molecular Devices).

### Determination of Collagen Content in Aortic Tissues

The wet weight of aortic tissues was determined. Tissues were then placed in 100 μl of a 0.5 M acetic acid solution and incubated at 65° C. for 5 hours to promote disintegration of the tissue. 20 μl of the resultant solution was loaded onto a 96 well plate along with collagen standards ranging from 12.5 μg to 200 μg. The plate was dried overnight at 55° C. and subsequently incubated with Picro-sirius Red as previously described.

### Measurement of Vascular Stiffness

Vessels were mounted on a steel wire myograph and incubated in Ca^{2±}-free Hank's Basic Salt Solution containing 100 μM sodium nitroprusside to ensure maximum relaxation. Vessels were then stretched incrementally to predefined tensions from 0.1 to 8 g. Images of the vessels at each incremental tension were captured and lumen diameters were measured with ImageJ software. Circumferential stress (a) was calculated as F/tl, where F is the tension in the vessel wall, t is the radial thickness of the vessel wall and 1 is the axial length of the aortic ring. Circumferential strain (c) was calculated as (D−D₀)/D₀ where D is the lumen diameter at a given tension and D₀ is the initial diameter under maximally relaxed conditions. The stress-strain data was fitted to an exponential equation σ=σ₀e^{ks}, where σ₀ is the initial stress in the relaxed vessel and k is a constant. Incremental elastic modulus was calculated from the first derivative of the stress-strain equation:

$$E = \frac{d σ}{d ϵ} = k {σ}_{0} {e}^{k ϵ} .$$

### Histology

Tissues were fixed in 4% paraformaldehyde and subsequently embedded in paraffin. 4 μm sections were cut, deparaffinized, and stained with Picro-Sirius Red for 1 hour, followed by two washes with 0.5% acetic acid. Slides were imaged using a light microscope and structural analyses were performed using ImageJ. Collagen area density was calculated by dividing the PSR-stained area by the total area of the vessel.

### Statistical Analysis

Values are expressed as mean±SEM. Statistical analyses were conducted using GraphPad Prism 6. For comparisons between the means of two groups, a Student's t-test was used. For comparisons of more than two groups, a one-way ANOVA was used, followed by a Newman-Keuls post hoc analysis for multiple comparisons. For analysis of data with two independent variables (treatment group and time), a two-way ANOVA was used.

### Results

### IRE1α Endonuclease Activity is Required for Collagen Synthesis in VSMCs and Fibroblasts.

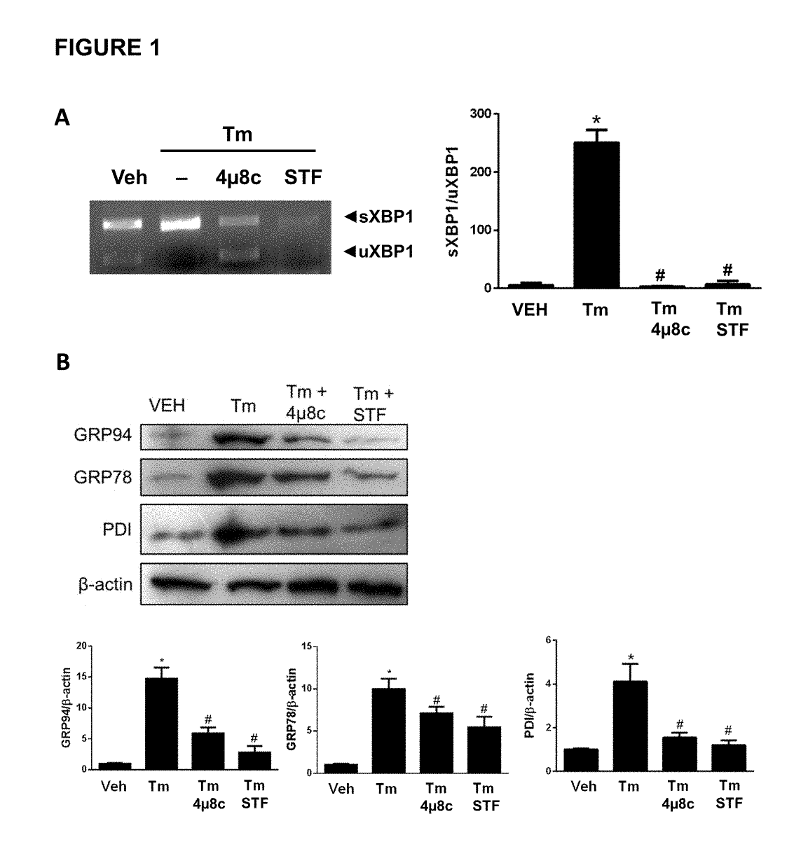

Aortic VSMCs were isolated from normotensive WKY rats and used for in vitro experiments. To determine the role of the IRE1α pathway in the synthesis of collagen by VSMCs, the IRE1α endonuclease inhibitors 4μ8c and STF-083010 were used. To test the ability of these compounds to block the IRE1α endonuclease, XBP1 splicing was measured in VSMCs treated with the ER stress inducer tunicamycin for 6 h with or without pre-treatment by 4μ8c or STF-083010. 4μ8c and STF-083010 reduced the ratio of spliced-to-unspliced)(BPI in cells treated with tunicamycin (FIG. 1A). 4μ8c and STF-083010 also reduced the elevated expression levels of ER stress markers GRP78, GRP94 and PDI after 24 h of tunicamycin treatment (FIG. 1B).

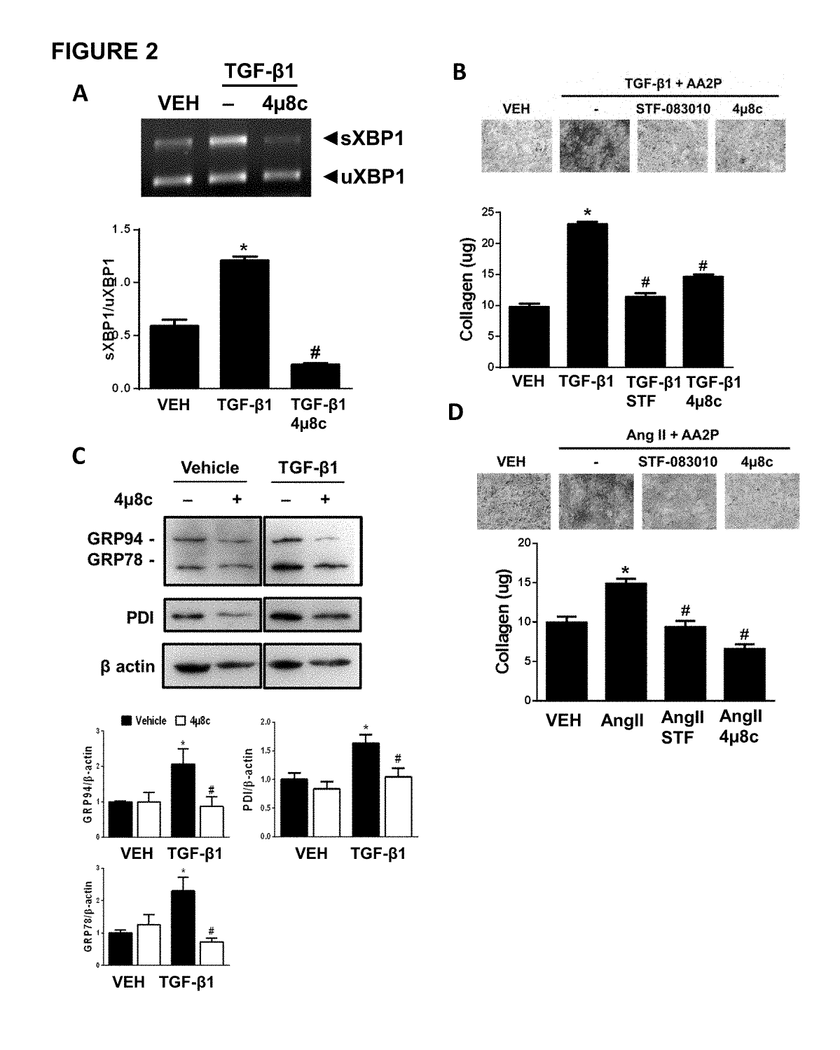

To demonstrate that IRE1α activation is necessary for collagen production in VSMCs, cells were treated with either TGF-β1 or Angiotensin II for 72 h, in the presence of L-ascorbic acid-2-phosphate (AA2P), to induce collagen synthesis. Cells were co-treated with either 4μ8c or S′TF-083010 to inhibit IRE1α endonuclease activity. TGF-β1 induced XBP1 splicing after 24 h, which was blocked by co-treatment with 4μ8c (FIG. 2A). Collagen production was measured using a Picro-Sirius Red-based colorimetric assay. TGF-β1 combined with AA2P induced collagen synthesis in the VSMCs, which was inhibited by co-treatment with 4μ8c or STF-083010 (FIG. 2B). TGF-β1 also increased the expression of the collagen chaperones and ER stress markers GRP78, GRP94 and PDI after 48 h, which was lowered by 4μ8c (FIG. 2C). 4μ8c also blocked collagen synthesis induced by Ang II (FIG. 2D).

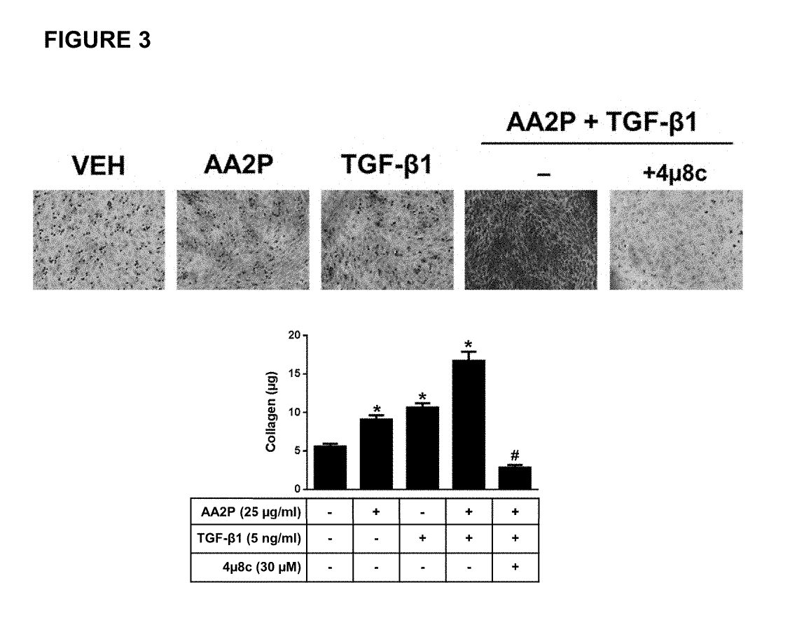

To determine if this was also true for fibroblasts, primary rat renal fibroblasts were used. Collagen levels were increased in response to TGF-β1 and reduced when co-treated with 4μ8c (FIG. 3A).

### Inhibition of IRE1α Endonuclease Activity Reduces Vascular Stiffening in Hypertension.

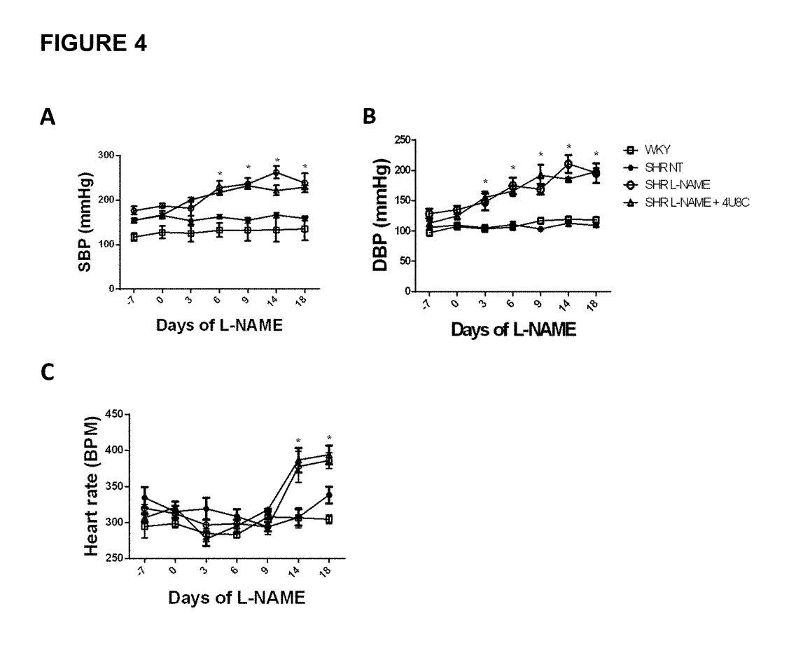

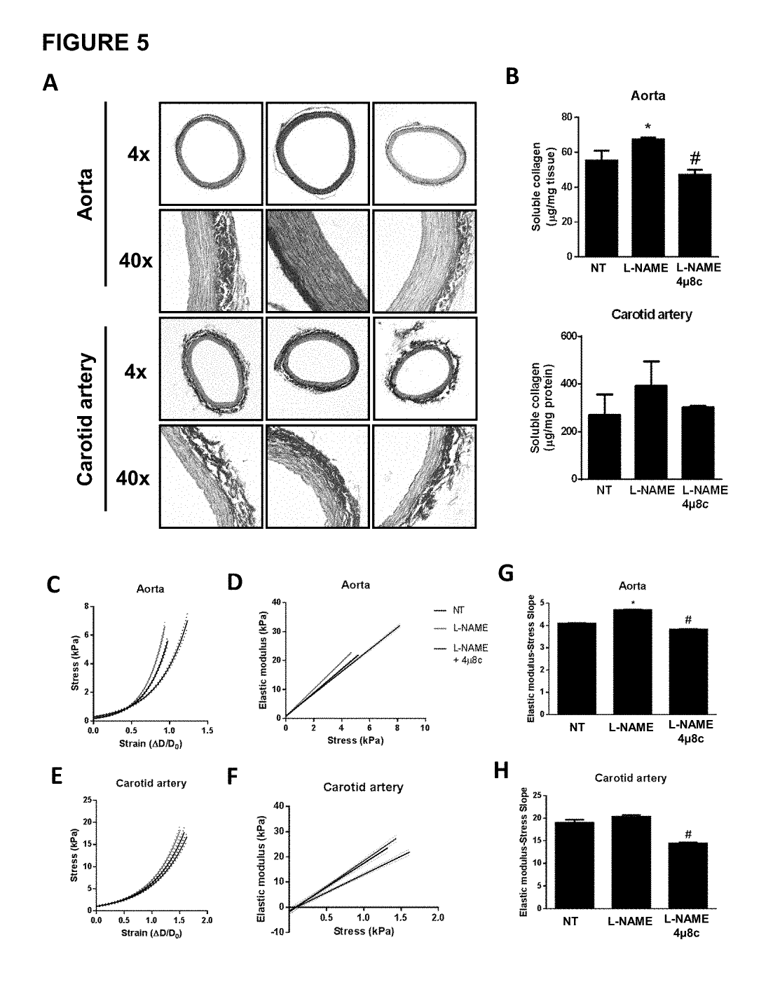

To demonstrate 4μ8c prevents vascular stiffening in hypertension, the SHR/L-NAME model of hypertension was used. 12-14 week old SHRs were implanted with radiotelemetry devices and treated with the nitric oxide synthase inhibitor L-NAME for 18 days. Rats were given daily injections of either vehicle or 4μ8c (2.5 mg/kg/day i.p.). Blood pressure was significantly increased in the SHR after 6 days of L-NAME treatment compared to baseline (FIG. 4). At Day 6, SBP had increased from 187.4±5.4 mmHg to 228.0±15.5 mmHg (p<0.05) and DBP from 134.8±6.5 mmHg to 174.5±13.6 mmHg. At Day 14, SBP and DBP reached a peak of 262.6±13.9 mmHg and 210.4±14.8 mmHg, respectively. Heart rate was also significantly elevated after 14 days of L-NAME treatment (377.6±21.4 bpm vs 312.7±3.0 bpm at baseline, p<0.05) and remained elevated until the end of the study (FIG. 4C). 4μ8c did not have an effect on the L-NAME-induced elevation in blood pressure or heart rate. In the L-NAME+4μ8c group, SBP increased from 166.6±8.4 mmHg at baseline to 216.7±4.8 mmHg at Day 6 while DBP increased from 124.7±5.5 mmHg at baseline to 166.2±8.2 mmHg at Day 6. SBP and DBP peaked at 232.3±5.4 mmHg and 192.1±17.0 mmHg, respectively, at Day 9. However, 4μ8c treatment reduced fibrosis and vascular stiffness in the hypertensive animals. Vessels were stained with PicroSirius Red and imaged using a light microscope (FIG. 5A). Collagen content in homogenized aortic tissue was measured using the PSR-based colorometric assay. Collagen content was significantly increased in the L-NAME group (67.31±1.09 μg/mg tissue) compared to the NT group (55.25±8.01 μg/mg tissue) and significantly reduced in the L-NAME+4μ8c group (47.10±2.88 μg/mg tissue).

Vascular stiffness of maximally relaxed vessels was measured using a wire myograph. Both the aorta and the carotid artery of L-NAME-treated SHRs demonstrated a leftward shift in the stress-strain relationship, indicative of increased vascular stiffness (FIGS. 5C and 5E). The slope of the incremental elastic modulus versus stress line was significantly increased in the aorta of the L-NAME group compared to the NT group (4.696±0.054 vs. 4.090±0.055, p<0.05) and significantly decreased in the L-NAME+4μ8c group compared to the L-NAME group (3.970±0.129, p<0.05) (FIGS. 5D and 5G). In the carotid artery, there was no difference in the slope of the elastic modulus-stress line between the L-NAME group and the NT group, but the slope of the L-NAME+4μ8c group was significantly lower compared to the other two groups (14.42±0.59 vs. 20.35±0.75 in the L-NAME group, p<0.05), indicative of reduced stiffness (FIGS. 5F and 5H).

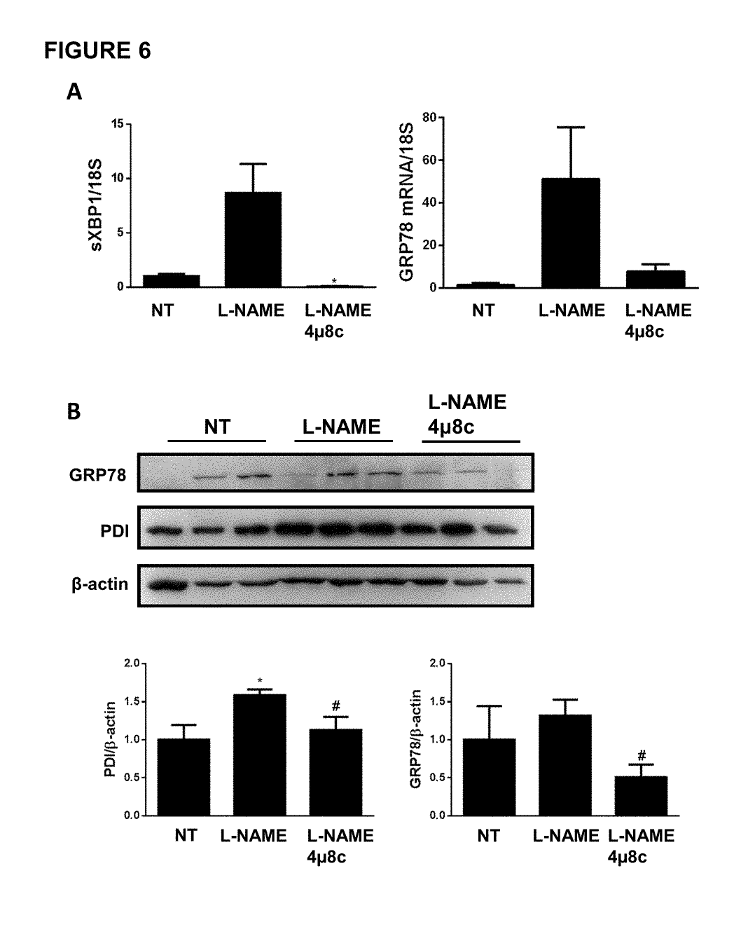

The expression of collagen-associated chaperones and ER stress markers in the aorta was analyzed by qRT-PCR and Western blotting. Spliced XBP1 and GRP78 mRNA expression were elevated in the L-NAME group and reduced in the L-NAME+4μ8c group (FIG. 6A). PDI protein expression was significantly increased in the L-NAME group and significantly decreased in the L-NAME+4μ8c group. GRP78 was significantly decreased in the L-NAME+4μ8c group compared to the other two groups.

### Inhibition of IRE1α Endonuclease Activity Reduces Cardiac Fibrosis in the SHR/L-NAME Model

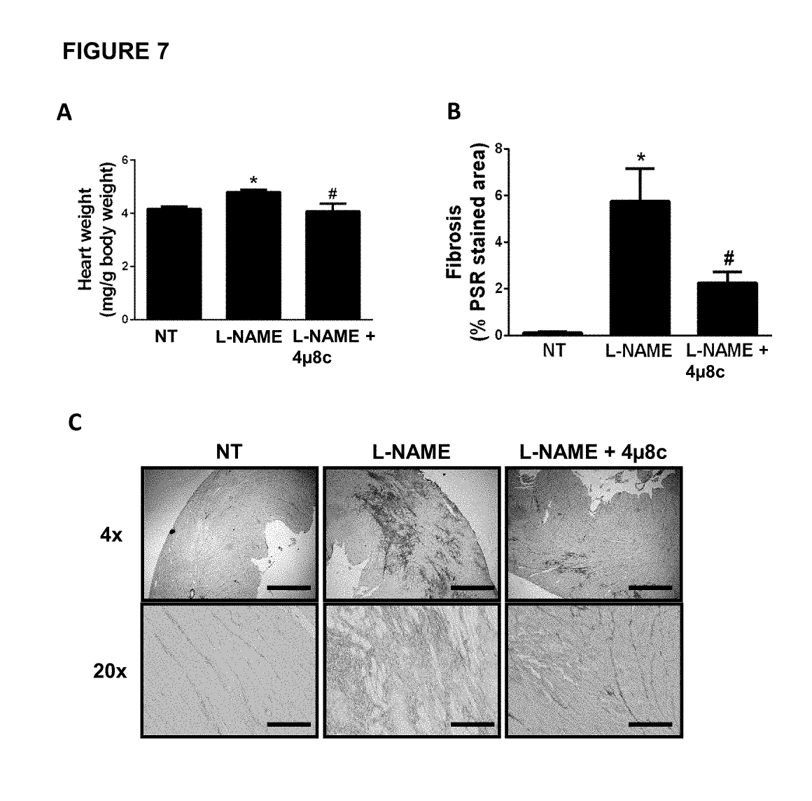

The SHR/L-NAME model is a model of hypertension-induced end-organ damage. Since vascular stiffness increases the risk of end-organ damage, it was hypothesized that 4μ8c would reduce the damage in the heart. Animals in the L-NAME+4μ8c group had significantly reduced cardiac hypertrophy compared to the L-NAME group (4.293±0.438 vs. 4.804±0.222 mg HW/g BW, p<0.05) (FIG. 7A). Animals in the L-NAME group developed severe fibrosis in the left and right ventricles as visualized by PSR staining, which was significantly reduced in the 4μ8c-treated animals (p<0.05) (FIGS. 7B and 7C).

### Discussion

Different embodiments of the invention have been shown by the above example. Those skilled in the art could develop alternatives to the methods mentioned above that are within the scope of the invention and defined claims.

### Effect of IRE1α Inhibition on Collagen Synthesis in VSMCs and Fibroblasts

Blocking IRE1α endonuclease activity with the small molecule inhibitors 4μ8c and STF-083010 prevented collagen secretion from primary rat VSMCs and fibroblasts. In the SHR/L-NAME model of malignant hypertension, daily injections of 4μ8c attenuated the development of cardiac fibrosis and vascular stiffening.

TGFβ1 and Ang II are known to stimulate collagen synthesis in VSMCs and fibroblasts^{15-22.} Here, the present inventors demonstrated that both molecules activated the IRE1α-XBP1s arm of the UPR in VSMCs after 24 h. Using these molecules in conjunction with L-ascorbic acid-phosphate (AA2P) resulted in collagen synthesis and deposition, which was measured by a Picro-sirius Red assay. Co-treatment with 4μ8c or STF-083010 inhibited collagen synthesis indicating that IRE1α endonuclease activity is likely required for the production, folding, or export of collagen molecules.

To elucidate the mechanisms by which IRE1α regulates collagen synthesis, the expression of ER resident chaperones that have been shown to interact with collagen during its biosynthesis were assessed. By qRT-PCR and Western blotting, the expression of GRP78, GRP94 and PDI were induced in ER stress conditions and lowered by IRE1α inhibition. Taken together, these results suggest that a mechanism by which IRE1α inhibition lowers collagen synthesis is by reducing the availability of ER-resident chaperones that are necessary for the proper folding of the procollagen triple helix.

### Effect of IRE1α Inhibition on Hypertension-Induced End-Organ Damage

The in vitro experiments performed in this study served as a proof of principle to justify the use of IRE1α inhibitors in animals undergoing active fibrotic remodelling processes. The overall aim of this study was to determine the role of the IRE1α pathway in the development of vascular stiffening during hypertension. Chronic inhibition of nitric oxide production with L-NAME resulted in the development of malignant hypertension in SHRs. This was associated with vascular stiffening¹², renal damage³¹⁻³³ and cardiac damage^{13,14}. The present inventors sought to determine whether systemic administration of the IRE1α inhibitor 4μ8c would prevent the development of fibrosis in this model. Animals were implanted with radiotelemetry devices for continuous measurement of blood pressure. As expected, L-NAME increased both systolic and diastolic blood pressures in the SHR. Daily injections of 4μ8c had no effect on the blood pressure elevation induced by L-NAME. This ruled out blood pressure as a confounder in any effects that were observed in terms of vascular stiffness or fibrosis. In both aorta and carotid blood vessels, treatment with 4μ8c reduced vascular stiffness even below the level of the non-treated control. These results demonstrate that IRE1α endonuclease activity is required for the development of vascular stiffening in hypertension and that the inhibitor, 4μ8c, prevents it.

The current therapeutic options for vascular stiffening and isolated systolic hypertension are limited to standard antihypertensive agents and lifestyle changes. Patients with ISH tend to be more resistant to antihypertensive treatments because the hypertension is driven by aortic stiffness³⁴. Thus, the development of more specific treatments that target vascular stiffening are warranted.

Herein it is demonstrated that the IRE1α pathway is required for collagen synthesis from VSMCs and fibroblasts and plays a role in the development of vascular stiffening and end-organ damage in hypertension. Inhibition of IRE1α endonuclease activity reduces the expression of chaperones involved in the biosynthesis of collagen, which hinders the proper folding and assembly of the collagen triple helix preventing vascular stiffening and end organ damage of the heart.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
IRE1-Alpha Inhibitors

| | | | CAS | | |
|---|---|---|---|---|---|
| Inhibitor | Synonym(s) | Formula | Number | Structure | Citation |
| STF- 083010; IRE1 Inhibitor I | N-[(2-Hydroxy-1- naphthyl)methylene]-2- thiophenesulfonamide, N- [(2-Hydroxynaphthalen-1- yl)methylidene]thiophene-2- sulfonamide | C₁₅H₁₁NO₃S₂ | 307543- 71-1 | 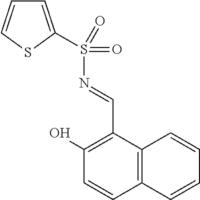 [SMILES: O=S(=O)(/N=C/c1c(O)ccc2ccccc12)c1cccs1] | Tufanli et al (2016), PNAS 28: 114(8): E1395- E1404 |
| IRE1 Inhibitor II | 3′-Formyl-4′-hydroxy-5′- methoxybiphenyl-3- carboxamide | C₁₅H₁₃NO₄ | 1093118- 73-0 | 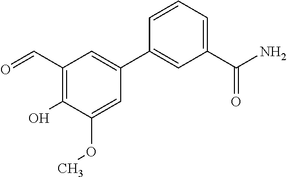 [SMILES: COc1cc(-c2cccc(C(N)=O)c2)cc(C=O)c1O] | WO 2008154484 A1 |
| CB5305630 | 7-Hydroxy-4-methyl-8- ((pyridine-2- ylimino)methyl)-2H- chromenone | C₁₆H₁₂N₂O₃ | 342388- 50-5 5 | 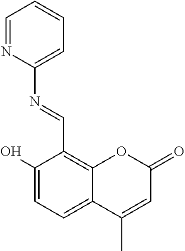 [SMILES: Cc1cc(=O)oc2c(/C=N/c3ccccn3)c(O)ccc12] | Tomasio et al, (2013) Molecular BioSystems, 9(10), 2408- 2416. |
| 4μ8C; IRE1 Inhibitor III | 7-Hydroxy-4-methyl-2-oxo- 2H-chromene-8- carbaldehyde | C₁₁H₈O₄ | 14003- 96-4 Structure 2 | 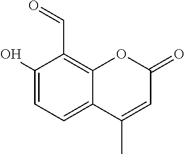 | Tuflani et al (see above) |
| KIRA6; IRE1 Inhibitor IV | 1-(4-(8-Amino-3-tert- butylimidazo[1,5-a]pyrazin- 1-yl)naphthalen-1-yl)-3-(3- (trifluoromethyl)phenyl)urea | C₂₈H₂₅F₃N₆O | 1589527- 65-0 | 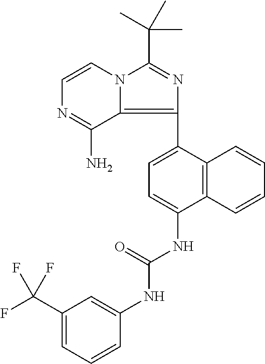 [SMILES: CC(C)(C)c1nc(-c2ccc(NC(=O)Nc3cccc(C(F)(F)F)c3)c3ccccc23)c2c(N)nccn12] | WO 2016004254 A1 |
| A-I06; IRE1 Inhibitor IV | 2-Hydroxy-naphthaldehyde | C₁₁H₈O₂ | 708-06-5 | 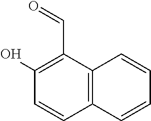 [SMILES: O=Cc1c(O)ccc2ccccc12] | US 20160083361 A1 |
| MKC- 3946; IRE1α Inhibitor | 2-Hydroxy-6-(5-(4- methylpiperazine-1- carbonyl)thiophen-2-yl)-1- naphthaldehyde | C₂₁H₂₀N₂O₃S | 1093119- 54-0 Structure 3 below | 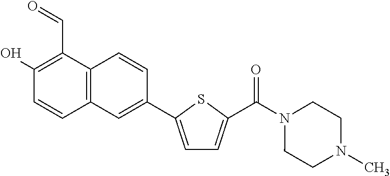 [SMILES: CN1CCN(C(=O)c2ccc(-c3ccc4c(C=O)c(O)ccc4c3)s2)CC1] | Ranatunga et al, (2014) Journal of Medicinal Chemistry, 57(10), 4289- 4301 |
| B-I09 | 7-(1,3-Dioxan-2-yl)-1,2,3,4- tetrahydro-8-hydroxy-5H- [1]benzopyrano[3,4- c]pyridin-5-one | C₁₆H₁₇NO₅ | 1607803- 67-7 | 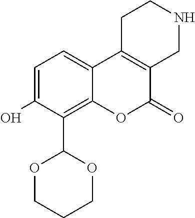 [SMILES: O=c1oc2c(C3OCCCO3)c(O)ccc2c2c1CNCC2] | US 20160083361 A1 |
| Compound 3 | 1-(4-(8-Amino-3- isopropylimidazo[1,5- a]pyrazin-1-yl)naphthalen-1- yl)-3-(3- (trifluoromethyl)phenyl)urea | C₂₇H₂₃F₃N₆O | 1414938- 21-8 | 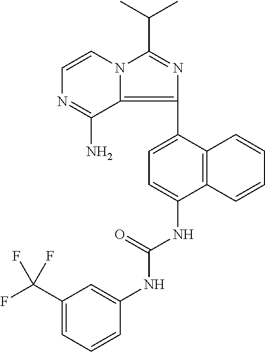 [SMILES: CC(C)c1nc(-c2ccc(NC(=O)Nc3cccc(C(F)(F)F)c3)c3ccccc23)c2c(N)nccn12] | WO 2016004254 A1 |
| Toyoca- mycin | 4-Amino-7-[3,4-dihydroxy- 5-(hydroxymethyl)oxolan-2- yl]pyrrolo[2,3-d]pyrimidine- 5-carbonitrile | C₁₂H₁₃N₅O₄ | 606-58-6 | 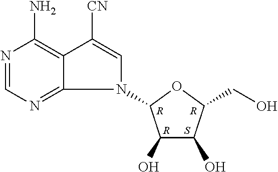 | US 20170165259 |
| Sunitinib | 1H-Pyrrole-3-carboxamide, N-[2-(diethylamino)ethyl]-5- [(Z)-(5-fluoro-1,2-dihydro- 2-oxo-3H-indol-3- ylidene)methyl]-2,4- dimethyl- | C₂₂H₂₇FN₄O₂ | 557795- 19-4 | 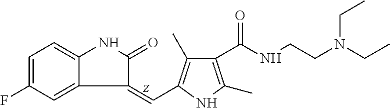 | WO 2016004254 A1 |
| APY29 | N2-1H-Benzimidazol-6-yl- N4-(5-cyclopropyl-1H- pyrazol-3-yl)-2,4- pyrimidinediamine | C₁₇H₁₆N₈ | 1216665- 49-4 | 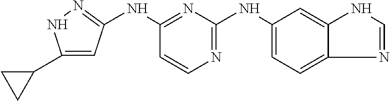 [SMILES: c1cc(Nc2cc(C3CC3)[nH]n2)nc(Nc2ccc3nc[nH]c3c2)n1] | WO 2016004254 A1 |
| Salicyl- aldehyde | 2-Hydroxybenzaldehyde | C₇H₆O₂ | 90-02-8 | 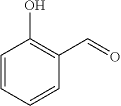 | WO 2016004254 A1 |
| Other salicyl- aldehyde derivatives | 3-Methoxy-6- bromosalicylaldehyde | C₈H₇BrO₃ | 20035- 41-0 | 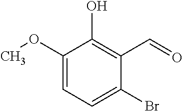 | WO 2016004254 A1 |

### REFERENCES

1. Harvey A, Montezano A C, Lopes R A, Rios F, Touyz R M. Vascular Fibrosis in Aging and Hypertension: Molecular Mechanisms and Clinical Implications. Can J Cardiol. 2016; 32:659-668.
2. Tritakis V, Tzortzis S, Ikonomidis I, Dima K, Pavlidis G, Trivilou P, Paraskevaidis I, Katsimaglis G, Parissis J, Lekakis J. Association of arterial stiffness with coronary flow reserve in revascularized coronary artery disease patients. World J Cardiol. 2016; 8:231-239.
3. Mattace-Raso F U S, Cammen T J M van der, Hofman A, Popele N M van, Bos M L, Schalekamp MADH, Asmar R, Reneman R S, Hoeks A P G, Breteler M M B, Witteman J C M. Arterial Stiffness and Risk of Coronary Heart Disease and Stroke. Circulation. 2006; 113:657-663.
4. Watanabe H, Ohtsuka S, Kakihana M, Sugishita Y. Coronary circulation in dogs with an experimental decrease in aortic compliance. J Am Coll Cardiol. 1993; 21:1497-1506.
5. Pase M P, Beiser A, Himali J J, Tsao C, Satizabal C L, Vasan R S, Seshadri S, Mitchell G F. Aortic Stiffness and the Risk of Incident Mild Cognitive Impairment and Dementia. Stroke. 2016; 47:2256-2261.
6. Hanon 0, Haulon S, Lenoir H, Seux M-L, Rigaud A-S, Safar M, Girerd X, Forette F. Relationship Between Arterial Stiffness and Cognitive Function in Elderly Subjects With Complaints of Memory Loss. Stroke. 2005; 36:2193-2197.
7. Sedaghat S, Mattace-Raso F U S, Hoorn E J, Uitterlinden A G, Hofman A, Ikram M A, Franco O H, Dehghan A. Arterial Stiffness and Decline in Kidney Function. Clin J Am Soc Nephrol. 2015; 10:2190-2197.
8. Madero M, Peralta C, Katz R, Canada R, Fried L, Najjar S, Shlipak M, Simonsick E, Lakatta E, Patel K, Rifkin D, Hawkins M, Newman A, Samak M, Study for the HA. Association of Arterial Rigidity with Incident Kidney Disease and Kidney Function Decline: The Health A B C Study. Clin J Am Soc Nephrol. 2013; 8:424-433.
9. Vlachopoulos C, Aznaouridis K, Stefanadis C. Prediction of Cardiovascular Events and All-Cause Mortality With Arterial Stiffness: A Systematic Review and Meta-Analysis. J Am Coll Cardiol. 2010; 55:1318-1327.
10. Tsamis A, Krawiec J T, Vorp D A. Elastin and collagen fibre microstructure of the human aorta in ageing and disease: a review. J R Soc Interface [Internet]. 2013 [cited 2016 Apr. 24]; 10. Available from: http://www.ncbi.nlm.nih.gov/pmc/articles/PMC3645409/11.
11. Maron B J. Hypertrophic Cardiomyopathy: A Systematic Review. JAMA. 2002; 287:1308-1320.
12. Isabelle M, Simonet S, Ragonnet C, Sansilvestri-Morel P, Clavreul N, Vayssettes-Courchay C, Verbeuren T J. Chronic reduction of nitric oxide level in adult spontaneously hypertensive rats induces aortic stiffness similar to old spontaneously hypertensive rats. J Vasc Res. 2012; 49:309-318.
13. Biwer L A, D′souza KM, Abidali A, Tu D, Siniard A L, DeBoth M, Huentelman M, Hale T_{M}. Time course of cardiac inflammation during nitric oxide synthase inhibition in SHR: impact of prior transient ACE inhibition. Hypertens Res Off J Jpn Soc Hypertens. 2016; 39:8-18.
14. Akashiba A, Ono H, Ono Y, Ishimitsu T, Matsuoka H. Valsartan improves 1-NAME-exacerbated cardiac fibrosis with TGF-β inhibition and apoptosis induction in spontaneously hypertensive rats. J Cardiol. 2008; 52:239-246.
15. Weigert C, Brodbeck K, Klopfer K, Haring H, Schleicher E. Angiotensin II induces human TGF-β1 promoter activation: similarity to hyperglycaemia. Diabetologia. 2002; 45:890-898.
16. Nataatmadja M, West J, Prabowo S, West M. Angiotensin II Receptor Antagonism Reduces Transforming Growth Factor Beta and Smad Signaling in Thoracic Aortic Aneurysm. Ochsner J. 2013; 13:42-48.
17. Ford C M, Li S, Pickering J G. Angiotensin II Stimulates Collagen Synthesis in Human Vascular Smooth Muscle Cells Involvement of the AT1 Receptor, Transforming Growth Factor-β, and Tyrosine Phosphorylation. Arterioscler Thromb Vasc Biol. 1999; 19:1843-1851.
18. Sorescu D. Smad3 Mediates Angiotensin II- and TGF-β1-Induced Vascular Fibrosis Smad3 Thickens the Plot. Circ Res. 2006; 98:988-989.
19. Schlumberger W, Thie M, Rauterberg J, Robenek H. Collagen synthesis in cultured aortic smooth muscle cells. Modulation by collagen lattice culture, transforming growth factor-beta 1, and epidermal growth factor. Arterioscler Thromb Vasc Biol. 1991; 11:1660-1666.
20. Kubota K, Okazaki J, Louie O, Kent K C, Liu B. TGF-β stimulates collagen (I) in vascular smooth muscle cells via a short element in the proximal collagen promoter. J Surg Res. 2003; 109:43-50.
21. Lu P, Wang S, Cai W, Sheng J. Role of TGF-β1/Smad3 Signaling Pathway in Secretion of Type I and III Collagen by Vascular Smooth Muscle Cells of Rats Undergoing Balloon Injury. J Biomed Biotechnol [Internet]. 2012 [cited 2015 Feb. 3]; 2012. Available from: http://www.ncbi.nlm.nih.gov/pmc/articles/PMC3471068/22.
22. Satoh C, Fukuda N, Hu W Y, Nakayama M, Kishioka H, Kanmatsuse K. Role of endogenous angiotensin II in the increased expression of growth factors in vascular smooth muscle cells from spontaneously hypertensive rats. J Cardiovasc Pharmacol. 2001; 37:108-118.
23. Acosta-Alvear D, Zhou Y, Blais A, Tsikitis M, Lents N H, Arias C, Lennon C J, Kluger Y, Dynlacht B D. XBP1 Controls Diverse Cell Type- and Condition-Specific Transcriptional Regulatory Networks. Mol Cell. 2007; 27:53-66.
24. Yoshida H, Matsui T, Yamamoto A, Okada T, Mori K. XBP1 mRNA Is Induced by ATF6 and Spliced by IRE1 in Response to ER Stress to Produce a Highly Active Transcription Factor. Cell. 2001; 107:881-891.
25. Van Duyn Graham L, Sweetwyne M T, Pallero M A, Murphy-Ullrich J E. Intracellular Calreticulin Regulates Multiple Steps in Fibrillar Collagen Expression, Trafficking, and Processing into the Extracellular Matrix. J Biol Chem. 2010; 285:7067-7078.
26. Ferreira L R, Norris K, Smith T, Hebert C, Sauk J J. Association of Hsp47, Grp78, and Grp94 with procollagen supports the successive or coupled action of molecular chaperones. J Cell Biochem. 1994; 56:518-526.
27. Duran I, Martin J H, Weis M A, Krejci P, Konik P, Li B, Alanay Y, Lietman C, Lee B, Eyre D, Cohn D H, Krakow D. A Chaperone Complex Formed by HSP47, FKBP65, and BiP Modulates Telopeptide Lysyl Hydroxylation of Type I Procollagen. J Bone Miner Res. 2017; n/a-n/a.
28. Wilson R, Lees J F, Bulleid N J. Protein disulfide Isomerase Acts as a Molecular Chaperone during the Assembly of Procollagen. J Biol Chem. 1998; 273:9637-9643.
29. Hall K L, Harding J W, Hosick H L. Isolation and characterization of clonal vascular smooth muscle cell lines from spontaneously hypertensive and normotensive rat aortas. Vitro Cell Dev Biol J Tissue Cult Assoc. 1991; 27A:791-798.
30. Xu Q, Norman J T, Shrivastav S, Lucio-Cazana J, Kopp J B. In vitro models of TGF-β-induced fibrosis suitable for high-throughput screening of antifibrotic agents. Am J Physiol—Ren Physiol. 2007; 293:F631-F640.
31. Ono H, Ono Y, Frohlich E D. ACE Inhibition Prevents and Reverses L-NAMEExacerbated Nephrosclerosis in Spontaneously Hypertensive Rats. Hypertension. 1996; 27:176-183.
32. Ono H, Ono Y, Frohlich E D. Nitric Oxide Synthase Inhibition in Spontaneously Hypertensive Rats Systemic, Renal, and Glomerular Hemodynamics. Hypertension. 1995; 26:249-255.
33. Zhou X, Ono H, Ono Y, Frohlich E D. Aldosterone Antagonism Ameliorates Proteinuria and Nephrosclerosis Independent of Glomerular Dynamics in L-NAME/SHR Model. Am J Nephrol. 2004; 24:242-9.
34. Smulyan H, Mookherjee S, Safar M E. The two faces of hypertension: role of aortic stiffness. J Am Soc Hypertens. 2016; 10:175-183.

What is claimed is:

1. A method of reducing fibrosis and/or collagen deposition in a subject, comprising administering an effective amount of an IRE1α inhibitor to the subject in need thereof.

2. A method of treating and/or reducing the risk of developing a condition associated with fibrosis and/or collagen deposition in a subject, comprising: administering an effective amount of an IRE1α inhibitor to the subject in need thereof.

3. The method of claim 2, wherein the condition associated with fibrosis and/or collagen deposition is aortic stiffening/hypertension, vascular fibrosis, vascular disease, cardiovascular disease, diastolic dysfunction, heart failure with preserved ejection fraction, arteriosclerosis, restenosis associated with vascular surgery and/or myocardial fibrosis.

4. The method of claim 3, wherein the condition associated with fibrosis and/or collagen deposition is aortic stiffening/hypertension.

5. The method of claim 2, wherein the condition associated with fibrosis and/or collagen deposition is interstitial renal fibrosis.

6. The method of claim 1, wherein the subject is human.

7. The method of claim 1, wherein the IRE1α inhibitor is a compound listed in Table 1 or an analog thereof.

8. The method of claim 7, wherein the IRE1α inhibitor is STF-083010, MKC-3946, 4μ8C, 3-methoxy-6-bromosalicylaldehyde, toyocamycin or an analog thereof.

9. The method of claim 7, wherein the IRE1α inhibitor is 4μ8C or an analog thereof.

10. The method of claim 7, wherein the IRE1α inhibitor is STF-083010 or an analog thereof.

11. The method of claim 1, wherein the IRE1α inhibitor is an antisense oligonucleotide molecule directed to IRE1α.

12. The method of claim 1, wherein the IRE1α inhibitor is an shRNA or siRNA molecule that inhibits expression of IRE1α.

13. The method of claim 1, wherein the IRE1α inhibitor is an antibody specific to IRE1α.

14. The method of claim 1, wherein the IRE1α inhibitor comprises a CRISPR-Cas9 complex to modulate the transcriptional activity at the endogenous IRE1α locus.

15. The method of claim 1, wherein the inhibitor is administered orally, topically or by local injection.
